(19) United States
(12) Patent Application Publication
(10) Pub. No.: US 2022/0211846 A1
(43) Pub. Date: Jul. 7, 2022

(54) ABT-165 IN COMBINATION WITH FOLINIC ACID, 5-FLUOROURACIL, AND IRINOTECAN FOR THE TREATMENT OF CANCERS

(71) Applicant: AbbVie Biotherapeutics Inc., Redwood City, CA (US)

(72) Inventor: Louie Naumovski, Los Altos, CA (US)

(21) Appl. No.: 17/403,930

(22) Filed: Aug. 17, 2021

Related U.S. Application Data
(63) Continuation of application No. 16/207,758, filed on Dec. 3, 2018, now abandoned.
(60) Provisional application No. 62/594,933, filed on Dec. 5, 2017.

(51) Int. Cl. A61K 39/395; C07K 16/28; C07K 16/22; A61K 31/519; A61K 31/4745; A61P 35/00; A61K 31/513

(52) U.S. Cl. CPC A61K 39/3955; C07K 16/28; C07K 16/22; A61K 2039/505; A61K 31/4745; A61P 35/00; A61K 31/513; A61K 31/519

(57) ABSTRACT

This invention pertains to a method for the treatment of cancer in a subject comprising administering to the subject an effective amount of ABT-165 in combination with folinic acid, 5-fluorouracil and irinotecan (FOLFIRI).

### RELATED APPLICATION

The application is a continuation of U.S. application Ser. No. 16/207,758, filed Dec. 3, 2018 which claims the benefit of U.S. Provisional Application Ser. No. 62/594,933, filed Dec. 5, 2017. The contents of each application are incorporated herein by reference in its entirety.

### FIELD OF THE INVENTION

This invention pertains to the use of ABT-165 in combination with folinic acid, 5-fluorouracil and irinotecan (FOLFIRI) in the treatment colorectal cancer, gastroesophageal cancer, pancreatic cancer, breast cancer, glioblastoma multiforme, ovarian cancer, or non-small cell lung cancer.

### BACKGROUND OF THE INVENTION

Delta-like ligand 4 (DLL4) is a cell surface-bound ligand that activates the Notch 1 receptor pathway, a conserved signaling cascade that regulates cell proliferation and cell fate determination (Gurney A, Hoey T. Vasc Cell. 2011; 3:18. Kuhnert F, Kirshner J R, and Thurston G. Vasc Cell. 2011; 3:20.). DLL4 is upregulated on tumor versus normal blood vessels (Jubb A M, Browning L, Campo L, et al. Histopathology. 2012; 60 (5):740-7. Jubb A M, Soilleux E J, Turley H, et al. Am J Pathol. 2010; 176 (4):2019-28. Jubb A M, Miller K D, Rugo H S, et al. Clin Cancer Res. 2011; 17 (2):372-81. Hu W, Lu C, Dong H H, et al. Cancer Res. 2011; 71 (18):6030-9.), playing a critical role in pathological angiogenesis, tumor initiating cell/cancer stem cell maintenance, and tumor cell chemoresistance through the interaction of the Notchl pathway and interplay with both vascular endothelial growth factor (VEGF)-dependent and -independent signaling (Gurney A, Hoey T. Vasc Cell. 2011;3:18. Kuhnert F, Kirshner J R, and Thurston G. Vasc Cell. 2011; 3:20. Noguera-Troise I, Daly C, Papadopoulos N J, et al. Nature. 2006; 444 (7122):1032-7. Ridgway J, Zhang G, Wu Y, et al. Nature. 2006; 444 (7122):1083-7. Hoey T, Yen W C, Axelrod F, et al. Cell Stem Cell. 2009; 5 (2):168-77. Yen W C, Fischer M M, Hynes M, et al. Clin Cancer Res. 2012; 18 (19):5374-86.).

VEGF is one of the best-studied regulators of tumor angiogenesis, playing key roles in both the establishment and survival of new tumor vasculature. VEGF binds and signals through vascular endothelial growth factor receptor (VEGFR)-2 that is upregulated on tumor endothelial cells engaged in angiogenesis (Hicklin D J, Ellis L M. J Clin Oncol. 2005; 23 (5):1011-27). Despite some of the proven clinical benefits of existing anti-VEGF therapies, both intrinsic and acquired patient resistance remain significant challenges, which highlight the need for better treatments that target VEGF-dependent and -independent pathways critical for tumor growth (Kerbel RS. N Engl J Med. 2008; 358 (19):2039-49.). Significant improvement beyond current therapy may be possible by developing combination approaches that target the DLL4 pathway in addition to the VEGF signaling axis, thereby targeting multiple mechanisms of action involved in tumor angiogenesis, tumor-initiating cell maintenance, and chemoresistance (Gurney A, Hoey T. Vasc Cell. 2011; 3:18. Ridgway J, Zhang G, Wu Y, et al. Nature. 2006; 444 (7122):1083-7. Li J L, Harris A L. Front Biosci. 2009; 14:3094-110.).

ABT-165 is a first-in-class humanized recombinant DVD-Ig molecule with a dual specificity for both human DLL4 and human VEGF. ABT-165 contains a human IgG1/κ isotype with two point mutations that diminish binding to Fcy receptors and complement component Clq, but demonstrates pH-dependent binding to FcRn within the expected range of human IgG1. ABT-165 exhibits a low ability to stimulate cytokine release by human peripheral blood cells (PBC) from normal donors and is within the expected range of other negative control IgG1 antibodies.

ABT-165 binds with nanomolar affinities to DLL4 and VEGF, and blocks DLL4 and VEGF interaction with their cognate receptors. As a result, it exhibits potent inhibition of DLL4-mediated Notch-1 activation, as well as inhibition of VEGF-stimulated endothelial cell proliferation. In preclinical animal studies, combined blockade of both DLL4 and VEGF resulted in increased inhibition of subcutaneous xenograft growth of human tumor cell lines derived from colon, breast, and glioblastoma, relative to blocking either axis alone. ABT-165 also induced tumor regression in vivo in combination with chemotherapy, with significantly better efficacy than the combination of anti-VEGF monoclonal antibody (mAb) therapy with cytotoxic agents.

ABT-165 has shown robust and reproducible antitumor effects in a variety of xenograft models including those derived from colorectal, breast, glioblastoma and pancreatic tumors. On the basis of unpublished, preclinical data, ABT-165 had demonstrated encouraging activity in tumors either as a single agent or in combination with standard of care (SOC) chemotherapeutics such as paclitaxel (Taxol®) or folinic acid (leucovorin), 5-fluorouracil, and irinotecan (FOLFIRI).

The present invention describes the clinical use of ABT-165 in combination with folinic acid (leucovorin), 5-fluorouracil, and irinotecan (FOLFIRI) in the treatment of colorectal cancer with significantly greater efficacy in human patients than otherwise anticipated from the preclinical experiments.

### BRIEF SUMMARY OF THE INVENTION

The present invention pertains to a method for the treatment of cancer comprising administering to the subject an effective amount of ABT-165 in combination with folinic acid, 5-fluorouracil, and irinotecan (FOLFIRI).

In one embodiment, the present invention pertains to a method for the treatment of colorectal cancer comprising administering to the subject an effective amount of ABT-165, or a pharmaceutically acceptable salt thereof, in combination with folinic acid (leucovorin), 5-fluorouracil, and irinotecan (FOLFIRI).

In one embodiment, the present invention pertains to a method for the treatment of breast cancer, glioblastoma multiforme, ovarian cancer, non-small cell lung cancer, gastroesophageal cancer, or pancreatic cancer comprising administering to the subject an effective amount of ABT-165, or a pharmaceutically acceptable salt thereof, in combination with folinic acid (leucovorin), 5-fluorouracil, and irinotecan (FOLFIRI).

In one embodiment, the present invention pertains to a method for the treatment of cancer that is sensitive to FOLFIRI therapy, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with folinic acid and 5-fluorouracil, wherein said cancer is selected from the group consisting of: gastroesophageal cancer, pancreatic cancer, breast cancer, glioblastoma multiforme, ovarian cancer, or non-small cell lung cancer in a subject who is in need thereof.

### BRIEF DESCRIPTION OF THE DRAWINGS

FIG. 1 shows superior efficacy of ABT-165 compared to an anti-VEGF monoclonal antibody in a colon cancer xenograft model. In this model, ABT-165 in combination with FOLFIRI also showed superior efficacy compared to an anti-VEGF monoclonal antibody in combination with FOLFIRI.

FIG. 2 shows a waterfall plot of second line (2 L) metastatic colorectal cancer (mCRC) patient results treated in a Phase 1b clinical trial treated with ABT-165/FOLFIRI. Partial response (PR) (≥30% change in the sum of the longest diameter of target tumor lesions) was achieved by 19% of the patients.

### DETAILED DESCRIPTION OF THE INVENTION

Multivalent and/or multispecific binding proteins capable of binding epitopes on two different proteins are provided. Dual variable domain binding proteins (also referred to as DVDs, DVD binding proteins, or dual variable domain immunoglobulins (DVD-Ig)), and pharmaceutical compositions thereof are provided.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting. Any range described here will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. To the extent documents incorporated by reference contradict the disclosure contained in the specification, the specification will supersede any contradictory material.

Generally, nomenclatures used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein unless otherwise indicated. The nomenclatures used in connection with, and the laboratory procedures and techniques of, analytical chemistry, synthetic organic chemistry, and medicinal and pharmaceutical chemistry described herein are those well-known and commonly used in the art unless otherwise indicated. Standard techniques are used for chemical syntheses, chemical analyses, pharmaceutical preparation, formulation, and delivery, and treatment of patients.

So that the disclosure may be more readily understood, select terms are defined below.

### Definitions

The terms “treat”, “treating” and “treatment” refer to a method of alleviating or abrogating a disease and/or its attendant symptoms.

The term “subject” is defined herein to include animals such as mammals, including, but not limited to, primates (e.g., humans). In preferred embodiments, the subject is a human.

The terms “patient” and “subject” are used herein interchangeably.

“Effective amount” refers to the amount sufficient to induce a desired biological, pharmacological, or therapeutic outcome in a subject. A therapeutically effective amount means a sufficient amount of a humanized recombinant DVD-Ig molecule with a dual specificity for both human DLL4 and human VEGF in combination with FOLFIRI to treat or prevent colorectal cancer, gastroesophageal cancer, pancreatic cancer, breast cancer, glioblastoma multiforme, ovarian cancer, or non-small cell lung cancer at a reasonable benefit/risk ratio applicable to any medical treatment. It will be understood, however, that the total usage of the compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compositions employed; the duration of the treatment; drugs used in combination or coincidental with the specific composition employed; and like factors well known in the medical arts.

The term “antibody” refers to an immunoglobulin (Ig) molecule, which is generally comprised of four polypeptide chains, two heavy (H) chains and two light (L) chains In an embodiment of a full-length antibody, each heavy chain is comprised of a heavy chain variable region (VH) and a heavy chain constant region (CH). The CH is comprised of three domains, CH1, CH2 and CH3. Each light chain is comprised of a light chain variable region (VL) and a light chain constant region (CL). The CL is comprised of a single CL domain. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG3, IgG4, IgA1 and IgA2), or subclass. For purposes of the present invention, the immunoglobulin is IgG1.

The term “humanized antibody” refers to an antibody from a non-human species that has been altered to be more “human-like”, i.e., more similar to human germline sequences. One type of humanized antibody is a CDR-grafted antibody, in which non-human CDR sequences are introduced into human VH and VL sequences to replace the corresponding human CDR sequences. A “humanized antibody” is also an antibody or a variant, derivative, analog or fragment thereof that comprises framework region (FR) sequences having substantially identity (e.g., at least 80%, at least 85%, at least 90%, at least 95%, at least 98% or at least 99% identity) to the amino acid sequence of a human antibody FR sequences and at least one CDR having substantial identity (e.g., at least 80%, at least 85%, at least 90%, at least 95%, at least 98% or at least 99% identity) to the amino acid sequence of a non-human CDR. A humanized antibody may comprise substantially all of at least one, and typically two, variable domains (Fab, Fab′, F(ab′)2, FabC, Fv) in which the sequence of all or substantially all of the CDR regions correspond to those of a non-human immunoglobulin (i.e., donor antibody) and the sequence of all or substantially all of the FR regions are those of a human immunoglobulin. The humanized antibody can also include the CH1, hinge, CH2, CH3, and CH4 regions of the heavy chain from a human antibody. In an embodiment, a humanized antibody also comprises at least a portion of a human immunoglobulin Fc region. In some embodiments, a humanized antibody only contains a humanized light chain. In some embodiments, a humanized antibody only contains a humanized heavy chain. In some embodiments, a humanized antibody only contains a humanized variable domain of a light chain and/or humanized variable domain of a heavy chain. In some embodiments, a humanized antibody contains a light chain as well as at least the variable domain of a heavy chain. In some embodiments, a humanized antibody contains a heavy chain as well as at least the variable domain of a light chain.

The terms “dual variable domain binding protein” and “dual variable domain immunoglobulin” refer to a binding protein that has two variable domains in each of its two binding arms (e.g., a pair of HC/LC) (see PCT Publication No. WO 02/02773), each of which is able to bind to an antigen. In an embodiment, each variable domain binds different antigens or epitopes. In another embodiment, each variable domain binds the same antigen or epitope. In another embodiment, a dual variable domain binding protein has two identical antigen binding arms, with identical specificity and identical CDR sequences, and is bivalent for each antigen to which it binds. In an embodiment, the DVD binding proteins may be monospecific, i.e., capable of binding one antigen or multi-specific, i.e., capable of binding two or more antigens. DVD binding proteins comprising two heavy chain DVD polypeptides and two light chain DVD polypeptides are referred to as a DVD-Ig. In an embodiment, each half of a four chain DVD binding protein comprises a heavy chain DVD polypeptide, and a light chain DVD polypeptide, and two antigen binding sites. In an embodiment, each binding site comprises a heavy chain variable domain and a light chain variable domain with a total of 6 CDRs involved in antigen binding per antigen binding site.

The term “biological activity” refers to any one or more biological properties of a molecule (whether present naturally as found in vivo, or provided or enabled by recombinant means). Biological properties include, but are not limited to, inhibiting tumor angiogenesis, inhibiting tumor-initiating/cancer stem cell maintenance, and inhibiting tumor cell chemoresistance.

The term “neutralizing” refers to counteracting the biological activity of an antigen when a binding protein specifically binds to the antigen. In an embodiment, a neutralizing binding protein binds to an antigen (e.g., a cytokine) and reduces its biologically activity by at least about 20%, 40%, 60%, 80%, 85% or more.

“Specificity” refers to the ability of a binding protein to selectively bind an antigen.

The term “potency” refers to the ability of a binding protein to achieve a desired effect, and is a measurement of its therapeutic efficacy. Potency may be assessed using methods known to one skilled in the art.

The term “biological function” refers the specific in vitro or in vivo actions of a binding protein. Binding proteins may target several classes of antigens and achieve desired therapeutic outcomes through multiple mechanisms of action. Binding proteins may target soluble proteins, cell surface antigens, and/or extracellular protein deposits. Binding proteins may agonize, antagonize, or neutralize the activity of their targets. Binding proteins may assist in the clearance of the targets to which they bind, or may result in cytotoxicity when bound to cells. Portions of two or more antibodies may be incorporated into a multivalent format to achieve more than one distinct function in a single binding protein molecule. in vitro assays and in vivo models used to assess biological function are known to one skilled in the art.

A “stable” binding protein is one in which the binding protein essentially retains its physical stability, chemical stability and/or biological activity upon storage. A multivalent binding protein that is stable in vitro at various temperatures for an extended period of time is desirable. Methods of stabilizing binding proteins and assessing their stability at various temperatures are known to one skilled in the art.

The term “solubility” refers to the ability of a protein to remain dispersed within an aqueous solution. The solubility of a protein in an aqueous formulation depends upon the proper distribution of hydrophobic and hydrophilic amino acid residues, and therefore, solubility can correlate with the production of correctly folded proteins. A person skilled in the art will be able to detect an increase or decrease in solubility of a binding protein using routine HPLC techniques and methods known to one skilled in the art.

The term “cytokine” refers to a protein released by one cell population that acts on another cell population as an intercellular mediator. The term “cytokine” includes proteins from natural sources or from recombinant cell culture and biologically active equivalents of the native sequence cytokines.

The term “component” refers to an element of a composition. In relation to a diagnostic kit, for example, a component may be a capture antibody, a detection or conjugate antibody, a control, a calibrator, a series of calibrators, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample. Thus, a “component” can include, in some embodiments, a polypeptide or other analyte as above, that is immobilized on a solid support, such as by binding to an anti-analyte (e.g., anti-polypeptide) antibody. In some embodiments, one or more components can be in solution or lyophilized.

“Control” refers to a composition that does not comprise an analyte (“negative control”) or does comprise the analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (e.g., analytes).

The term “Fc region” defines the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (e.g., U.S. Pat. Nos. 5,648,260 and 5,624,821). The Fc region mediates several important effector functions, e.g., cytokine induction, antibody dependent cell mediated cytotoxicity (ADCC), phagocytosis, complement dependent cytotoxicity (CDC), and the half-life/clearance rate of antibody and antigen-antibody complexes. In some cases these effector functions are desirable for a therapeutic immunoglobulin but in other cases might be unnecessary or even deleterious, depending on the therapeutic objectives.

The term “multivalent binding protein” means a binding protein comprising two or more antigen binding sites. In an embodiment, the multivalent binding protein is engineered to have three or more antigen binding sites, and is not a naturally occurring antibody. The term “multi-specific binding protein” refers to a binding protein capable of binding two or more related or unrelated targets. In an embodiment, the DVD binding proteins provided herein comprise two or more antigen binding sites and are tetravalent or multivalent binding proteins.

The term “linker” means an amino acid residue or a polypeptide comprising two or more amino acid residues joined by peptide bonds that are used to link two polypeptides (e.g., two VH or two VL domains). Examples of such linker polypeptides are well known in the art (see, e.g., Holliger et al. (1993) Proc. Natl. Acad. Sci. USA 90: 6444-6448; Poljak et al. (1994) Structure 2:1121-1123).

The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad. Sci. 190: 382-391 and, Kabat et al. (1991) Sequences of Proteins of Immunological Interest, Fifth Edition, U.S. Department of Health and Human Services, NIH Publication No. 91-3242).

The term “CDR” means a complementarity determining region within an immunoglobulin variable region sequence. There are three CDRs for each epitope in each of the variable regions of the heavy chain and the light chain, which are designated CDR1, CDR2 and CDR3, for each of the heavy and light chain variable regions. The term “CDR set” refers to a group of three CDRs that occur in a single variable region capable of binding the antigen. The exact boundaries of these CDRs have been defined differently according to different systems. The system described by Kabat (Kabat et al. (1987) and (1991)) not only provides an unambiguous residue numbering system applicable to any variable region of an antibody, but also provides precise residue boundaries defining the three CDRs. These CDRs may be referred to as Kabat CDRs. Chothia and colleagues (Chothia and Lesk (1987) J. Mol. Biol. 196:901-917; Chothia et al. (1989) Nature 342:877-883) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262 (5):732-45). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The term “epitope” means a region of an antigen that is bound by a binding protein, e.g., a region capable of specifically binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In an embodiment, an epitope comprises the amino acid residues of a region of an antigen (or fragment thereof) known to bind to the complimentary site on the specific binding partner. An antigenic fragment can contain more than one epitope. In certain embodiments, a binding protein specifically binds an antigen when it recognizes its target antigen in a complex mixture of proteins and/or macromolecules. Binding proteins “bind to the same epitope” if the antibodies cross-compete (e.g., one prevents the other from binding to the binding protein, or inhibits the modulating effect on the other of binding to the binding protein). The methods of visualizing and modeling epitope recognition are known to one skilled in the art (US 20090311253).

The term “variant” means a polypeptide that differs from a given polypeptide in amino acid sequence by the addition (e.g., insertion), deletion, or conservative substitution of amino acids, but that retains the biological activity of the given polypeptide (e.g., a variant VEGF antibody can compete with anti-VEGF antibody for binding to VEGF). A conservative substitution of an amino acid, i.e., replacing an amino acid with a different amino acid of similar properties (e.g., hydrophilicity and/or degree or distribution of charged regions) is recognized in the art as typically involving a minor change. These minor changes can be identified, in part, by considering the hydropathic index of amino acids, as understood in the art (see, e.g., Kyte et al. (1982) J. Mol. Biol. 157: 105-132). In one aspect, amino acids having hydropathic indexes of ±2 are substituted. The hydrophilicity of amino acids can also be used to reveal substitutions that would result in proteins that retain biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” also includes polypeptides or fragments thereof that have been differentially processed, such as by proteolysis, phosphorylation, or other post-translational modification, yet retain biological activity and/or antigen reactivity, e.g., the ability to bind to VEGF and/or DLL4. The term “variant” encompasses fragments of a variant unless otherwise defined. A variant may be 99%, 98%, 97%, 96%, or 95%identical to the wild type sequence.

“Folinic acid” is (2S)-2-(4-{[(2-amino-5-formyl-4-oxo-1,4,5,6,7,8-hexahydropteridin-6-yl)methyl]amino}benzamido)pentanedioic acid and has CAS No. 1492-18-8. Folinic acid is also known as leucovorin and 5-formyltetrahydrofolate. The molecular formula is C₂₀H₂₃N₇O₇ and the molecular weight is 473.44 g/mol.

“Irinotecan” is (4S)-4,11-diethyl-4-hydroxy-3,14-dioxo-3,4,12,14-tetrahydro-1H-pyrano[3′,4′:6,7]indolizino[1,2-b]quinolin-9-yl [1,4′-bipiperidine]-1′-carboxylate and has CAS No. 97682-44-5. Irinotecan is also known by the trade names Camptosar (US) and Campto (EU). The molecular formula is C₃₃H₃₈N₄O₆ and the molecular weight is 586.678.

The term “FOLFOX” means a chemotherapeutic comprising folinic acid, 5-fluorouracil, and oxaliplatin.

The term “FOLFIRI” means a chemotherapeutic regimen comprising folinic acid, 5-fluorouracil, and irinotecan.

In one embodiment, the present invention relates to ABT-165, a dual-variable domain humanized immunoglobulin molecule with dual specificity for both delta-like ligand 4 (DLL4) and vascular endothelial growth factor (VEGF). It is disclosed as a dual variable domain immunoglobulin, h1A11.1-SL-Av, in U.S. Pat. No. 9,163,093B2, incorporated herein by reference in its entirety and for all purposes.

In one embodiment, the present invention relates to ABT-165, a dual-variable domain immunoglobulin molecule with dual specificity for both delta-like ligand 4 (DLL4) and vascular endothelial growth factor (VEGF) consisting of 2 light chain protein binding sequences of SEQ ID No. 15 and 2 heavy chain protein binding sequences of SEQ ID No. 16. ABT-165 may also be described as a dual-variable domain immunoglobulin molecule comprising light chain CDRs of SEQ ID Nos. 1-3 with affinity for DLL4, light chain CDRs of SEQ ID Nos. 4-6 with affinity for VEGF, heavy chain CDRs of SEQ ID Nos. 7-9 with affinity for DLL4, and heavy chain CDRs of SEQ ID Nos. 10-12 with affinity for VEGF.

In one embodiment, the present invention relates to a method for the treatment of colorectal cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with folinic acid, 5-fluorouracil, and irinotecan (FOLFIRI) as first line therapy.

In one embodiment, the present invention relates to a method for the treatment of colorectal cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg every 2 weeks in combination with folinic acid, 5-fluorouracil, and irinotecan (FOLFIRI) as first line therapy.

In one embodiment, the present invention relates to a method for the treatment of colorectal cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with folinic acid, 5-fluorouracil, and irinotecan (FOLFIRI) as second line or higher therapy.

In one embodiment, the present invention relates to a method for the treatment of colorectal cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg every 2 weeks in combination with folinic acid, 5-fluorouracil, and irinotecan (FOLFIRI) as second line or higher therapy.

In one embodiment, the present invention relates to a method for the treatment of colorectal cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with folinic acid, 5-fluorouracil, and irinotecan (FOLFIRI) as first line therapy or second line or higher therapy.

In one embodiment, the present invention relates to a method for the treatment of colorectal cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg every 2 weeks in combination with folinic acid, 5-fluorouracil, and irinotecan (FOLFIRI) as first line therapy or second line or higher therapy.

FOLFIRI (folinic acid, 5-fluorouracil, and irinotecan) is indicated for the treatment of colorectal cancer as well as gastric cancer. Other cancers may also be sensitive to FOLFIRI, and thus may be amenable to treatment comprising the combination of ABT-165 and FOLFIRI.

Avastin® (bevacizumab) is indicated for the treatment of metastatic colorectal cancer, non-squamous non-small cell lung cancer, glioblastoma, metastatic renal cell carcinoma, metastatic cervical cancer, ovarian cancer, fallopian tube cancer, and peritoneal cancer. These cancers may also be sensitive to treatment comprising the combination of ABT-165 and FOLFIRI.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with at least one additional cancer agent.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination once every 2 weeks with at least one additional cancer agent.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with irinotecan.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg every 2 weeks in combination with irinotecan.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with 5-fluorouracil and folinic acid.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg every 2 weeks in combination with 5-fluorouracil and folinic acid.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with 5-fluorouracil and irinotecan.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg every 2 weeks in combination with 5-fluorouracil and irinotecan.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with folinic acid, 5-fluorouracil, and irinotecan (FOLFIRI).

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg every 2 weeks in combination with folinic acid, 5-fluorouracil, and irinotecan (FOLFIRI).

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with folinic acid, 5-fluorouracil, and irinotecan (FOLFIRI) as first line (1L) therapy.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg every 2 weeks in combination with folinic acid, 5-fluorouracil, and irinotecan (FOLFIRI) as first line therapy.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with folinic acid, 5-fluorouracil, and irinotecan (FOLFIRI) as second line or higher therapy.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg every 2 weeks in combination with folinic acid, 5-fluorouracil, and irinotecan (FOLFIRI) as second line or higher therapy.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with folinic acid, 5-fluorouracil, and irinotecan (FOLFIRI) as first line therapy or second line or higher therapy.

In one embodiment, the present invention relates to a method for the treatment of colorectal cancer, gastroesophageal cancer, pancreatic cancer, breast cancer, glioblastoma multiforme, ovarian cancer, or non-small cell lung cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg every 2 weeks in combination with folinic acid, 5-fluorouracil, and irinotecan (FOLFIRI) as first line therapy or second line or higher therapy.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Throughout this application, the term “about” is used to indicate that a value includes the inherent variation of error for the device, the method being employed to determine the value, or the variation that exists among the study subjects. When used in the context of dosing, the term “about” is used to indicate a value of ±10% from the reported value, preferably a value of ±5% from the reported value.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In embodiments, complementarity determining regions (CDRs) are provided, directed against epitopes of DLL4 or VEGF on either a variable light (VL) or variable heavy (VH) binding protein. The amino acid sequences of theses CDRs are shown below in Table 1.

TABLE 1
CDRs of variable light (VL) chain and
variable heavy (VH) binding proteins
directed to epitopes of DLL4 or VEGF.

| SEQ ID No. | Protein region | Sequence |
|---|---|---|
| 1 | DLL4 VL | RASEDIYSNLA |
| 2 | DLL4 VL | DTNNLAD |
| 3 | DLL4 VL | QQYNNYPPT |
| 4 | VEGF VL | SASQDISNYLN |
| 5 | VEGF VL | FTSSLHS |
| 6 | VEGF VL | QQYSTVPW |
| 7 | DLL4 VH | GFTFSNFPMA |
| 8 | DLL4 VH | ISSSDGTTYYRDSVKG |
| 9 | DLL4 VH | GYYNSPFAY |
| 10 | VEGF VH | SGYTFTNYGMN |
| 11 | VEGF VH | INTYTGEPTYAADFKR |
| 12 | VEGF VH | YPHYYGSSHWYFDV |

In an embodiment, a DVD binding proteins is provided, comprising a variable light (VL) region, SEQ ID No. 13. In another embodiment, a DVD binding protein is provided, comprising a variable heavy (VH) region, SEQ ID No. 14. The amino acid sequences for these VL and VH domains are shown below in Table 2.

TABLE 2
Variable light (VL) and variable heavy (VH) DVD
binding proteins of ABT-165 directed against
epitopes of DLL4 and VEGF. (Linker sequences
are italicized and CDR sequences are bolded.).

| SEQ | ABBV | |
|---|---|---|
| ID No. | Unique ID | Sequence |
| 13 | h1A11.1- | DIQMTQSPSSLSASVGDRVTITCRASEDIYSN |
| | SL-Av | LAWYQQKPGKAPKLLIYDTNNLADGVPSRFSG |
| | VL | SGSGTDFTLTISSLQPEDFATYYCQQYNNYPP |
| | | TFGQGTKLEIKRTVAASPVFIFPPDIQMTQSP |
| | | SSLSASVGDRVTITCSASQDISNYLNWYQQKP |
| | | GKAPKVLIYFTSSLHSGVPSRFSGSGSGTDFT |
| | | LTISSLQPEDFATYYCQQYSTVPWTFGQGTKV |
| | | EIKR |
| 14 | h1A11.1- | EVQLVESGGGLVQPGGSLRLSCAASGFTFSNF |
| | SL-Av | PMAWVRQAPGKGLEWVATISSSDGTTYYRDSV |
| | VH | KGRFTISRDNAKNQLYLQMNSLRAEDTAVYYC |
| | | ARGYYNSPFAYWGQGTLVTVSSASKTGPEVQL |
| | | VESGGGLVQPGGSLRLSCAASGYTFTNYGMNW |
| | | VRQAPGKGLEWVGWINTYTGEPTYAADFVKRR |
| | | FTFSLDTSKSTAYLQMNSLRAEDTAVYYCAKY |
| | | PHYYGSSHWYFDVWGQGLTVTVSS |

Table 3 provides the full-length heavy and light chain sequences for binding proteins directed against VEGF and DLL4 in ABT-165.

TABLE 3
Full length light chain and heavy chain
binding proteins of ABT-165 directed against
epitopes of DLL4 and VEGF. (Linker sequences
are italicized, constant region sequences
are underlined, and CDRs are bolded.)

| SEQ | ABBV | |
|---|---|---|
| ID No. | Unique ID | Sequence |
| 15 | h1A11.1- | DIQMTQSPSSLSASVGDRVTITCRASEDIYS |
| | SL-Av | NLAWYQQKPGKAPKLLIYDTNNLADGVPSRF |
| | light | SGSGSGTDFTLTISSLQPEDFATYYCQQYNN |
| | chain | YPPTFGQGTKLEIKRTVAAPSVFIFPPDIQM |
| | | TQSPSSLSASVGDRVTITCSASQDISNYLNW |
| | | YQQKPGKAPKVLIYFTSSLHSGVPSRFSGSG |
| | | SGTDFTLTISSLQPEDFATYYCQQYSTVPWT |
| | | FGQGTKVEIKRTVAAPSVFIFPPSDEQLKSG |
| | | TASVVCLLNNFYPREAKVQWKVDNALQSGNS |
| | | QESVTEQDSKDSTYSLSSTLTLSKADYEKHK |
| | | VYACEVTHQGLSSPVTKSFNRGEC |
| 16 | h1A11.1- | EVQLVESGGGLVQPGGSLRLSCAASGFTFSN |
| | SL-Av- | FPMAWVRQAPGKGLEWVATISSSDGTTYYRD |
| | heavy | SVKGRFTISRDNAKNSLYLQMNSLRAEDTAV |
| | chain | YYCARGYYNSPFAYWGQGTLVTVSSASTKGP |
| | | EVQLVESGGGLVQPGGSLRLSCAASGYTFTN |
| | | YGMNWVRQAPGKGLEWVGWINTYTGEPTYAA |
| | | DFKRRFTFSLDTSKSTAYLQMSLRAEDTAVY |
| | | YCAKYPHYYGSSHWYFDVWGQGLTVTVSSAS |
| | | KTGPSVFPLAPSSKSTSGGTAALGCLVKDYF |
| | | PEPVTVSWNSGALTSGVHTFPAVLQSSGLYS |
| | | LSSVVTVPSSSLGTQTYICNVNHKPSNTKVD |
| | | KKVEPKSCDKTHTCPPVPAPEAAGGPSVFLF |
| | | PPKPKDTLMISRTPEVTCVVVDVSHEDPEVK |
| | | FNWYVDGVEVHNAKTKPREEQYNSTYRVVSV |
| | | LTVLHQDWLNGKEYKCKVSNKALPAPIEKTI |
| | | SKAKGQPREPQVYTLPPSREEMTKNQVSLTC |
| | | LVKGFYPSDIAVEWESNGQPENNYKTTPPVL |
| | | DSDGSFFLYSKLTVDKSRWQQGNVFSCSVMH |
| | | EALHNHY6TQKSLSLSPGK |

### BRIEF DESCRIPTION OF THE SEQUENCE LISTING

Incorporated herein by reference in its entirety is a Sequence Listing entitled, “ABV12424USL1 SEQ ID LIST_ST25”, comprising SEQ ID NO: 1 through SEQ ID NO: 16, which includes the amino acid sequences disclosed herein. The Sequence listing has been submitted herewith in ASCII text format via EFS. The Sequence Listing was first created on Dec. 4, 2017, and is 15 KB in size.

### EXAMPLES

### Example 1

### HCT-116 Colon Xenograft Model

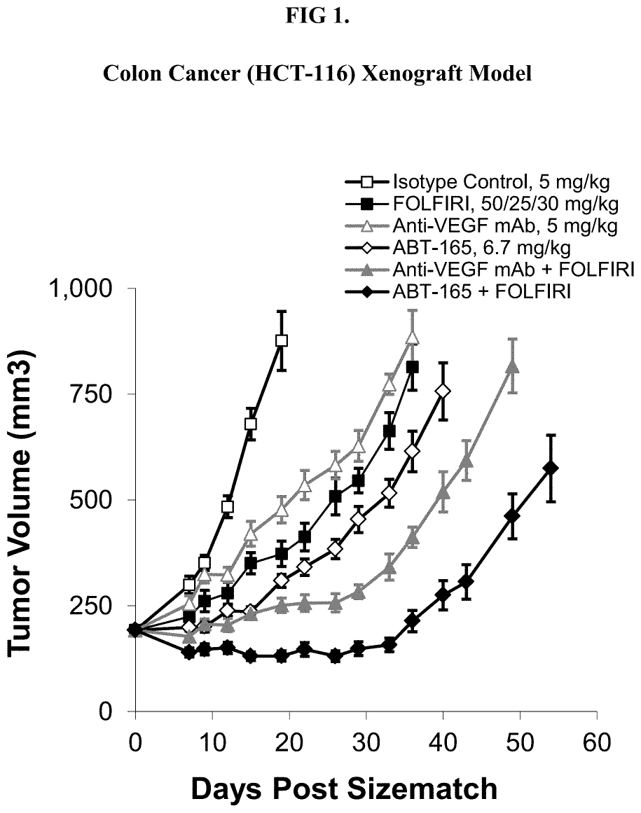

The effect of anti-DLL4-VEGF DVD-Igs (ABT-165) in combination with chemotherapy on tumor growth was evaluated on HCT-116 human colon xenograft tumors in female SCID mice. Briefly, 5×10⁶ cells were inoculated subcutaneously into the right hind flank. Tumors were allowed to establish for 14 days, at which point tumor volume was determined using electronic caliper measurements using the formula: L×W²/2. Mice were allocated into treatment groups (n=9 per group) so that each cohort had equivalent mean tumor volume of 192 mm³ prior to initiation of therapy. Animals were dosed with 5-fluorouracil (5-FU), folinic acid (leucovorin), irinotecan, anti-VEGF mAb (AB014), and/or anti-DLL4-VEGF DVD-Ig (ABT-165) at the dose and schedule in Table 4. An anti-tetanus toxoid mAb (AB095) was used as a non-targeting isotype control. Tumor volume was measured twice a week for the duration of the experiment. Results are shown in Table 4 and FIG. 1.

TABLE 4
Combination efficacy of anti-DLL4-VEGF DVD-Ig (ABT-165)
and FOLFIRI in the HCT-116 colon xenograft model

| Treatment | Dose Route, Regimen | % TGI^{a} |
|---|---|---|
| folinic acid | 25 mg/kg PO, q7d × 3 | 63* |
| 5-fluorouracil | 50 mg/kg IV, q7d × 3 | |
| irinotecan (FOLFIRI) | 30 mg/kg IV, q7d × 3 | |
| Anti-VEGF mAb | 5 mg/kg IP, q7d × 4 | 49* |
| ABT-165 | 6.7 mg/kg IP, q7d × 4 | 67* |
| Anti-VEGF mAb + | 5 mg/kg IP, q7d × 4 + | 81* |
| FOLFIRI | (above) q7d × 3 | |
| ABT-165 + FOLFIRI | 6.7 mg/kg IP, q7d × 4 + | 90* |
| | (above) q7d × 3 | |

Table 4 key. ^{a}% TGI = Percent tumor growth inhibition = 100 − (T/C × 100), where T = mean tumor volume of treatment group and C = mean tumor volume of treatment control group. Based on day 26 post size match measurements.
P values (as indicated by asterisks) are derived from Student's T test comparison of treatment group vs. treatment control group: * p < 0.0001.
“q7d × 3” indicates administration every seven days for three cycles (i.e., 3 doses),
while “q7d × 4” indicates administration every seven days for four cycles.

### Example 2

### Study of ABT-165 in Subjects with Advanced Solid Tumors

Sixteen subjects with second line (2 L) metastatic colorectal cancer (mCRC) were treated with a combination of ABT-165 at 2.5 mg/kg given every 14 days intravenously and the chemotherapy FOLFIRI which consists of folinic acid (leucovorin) [dl-400 mg/m² over 120 minutes or 1-200 mg/m² over 120 minutes], 5-fluorouracil [400 mg/m² IV bolus followed by 2400 mg/m² via IV infusion over 46 to 48 hours], and irinotecan [180 mg/m² over 90 minutes] in a Phase 1b study. Most of the 16 subjects had previously received a FOLFOX (5-fluorouracil, folinic acid (leucovorin) and oxaliplatin) regimen along with bevacizumab, an anti-vascular endothelial growth factor (VEGF) antibody as standard first line therapy for mCRC. Subjects were treated until their cancer progressed or until they could no longer tolerate the therapy due to toxicity. Tumor assessments consist of computerized tomography (CT) scans (or magnetic resonance imaging (MM) where clinically indicated) and were performed approximately every 8 weeks and compared to the baseline scan prior to treatment with ABT-165/FOLFIRI.

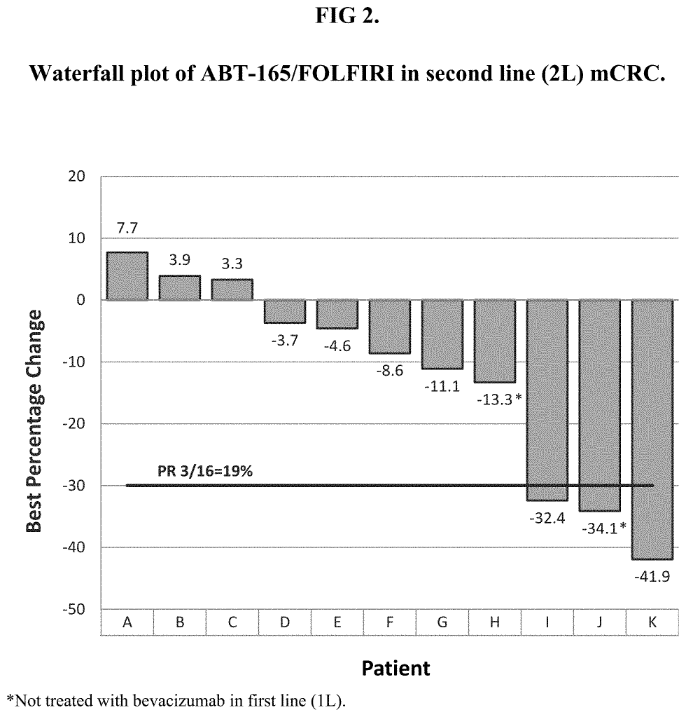

The waterfall plot (FIG. 2) shows the “best percentage change” in tumor measurements (sum of the longest diameter of target lesions as per Response Evaluation Criteria in Solid Tumors (RECIST, Version 1.1). As of data cut-off, tumor measurements were available for 11 subjects of the 16 subjects treated; no data was available for 5 subjects either because they did not yet have a scan or never had a scan prior to discontinuing from the study. Considering all the subjects treated, 3 out of 16 (19%) had a partial response (PR) and 8 out of 16 (50%) had stable disease by RECIST. Two of the 3 subject who had a PR were previously treated with FOLFOX+bevacizumab. Two of the 3 subjects who had a partial response were noted to have >30% decrease in the sum of the longest diameter of target lesions on at least 2 CT scans separated by ˜8 weeks and therefore qualify as “confirmed” partial response.

Historical data from a prior study (Bennouna J, Sastre J, et al. Lancet Oncol. 2013; 14:29-37) showed that the partial response (PR) rate in second line (2 L) mCRC subjects previously treated with a first line (1 L) chemotherapy (oxaliplatin or irinotecan based) plus bevacizumab regimen was only ˜5%. Hence, the data from the Phase 1b combination study of ABT-165+FOLFIRI in 2 L mCRC subjects, as shown in FIG. 2, demonstrated considerably higher anti-tumor response for the ABT-165+FOLFIRI combination (19%) than that shown in the historical bevacizumab+chemotherapy study (˜5%). The benefit provided by the combination of ABT-165+FOLFIRI was unexpected from prior clinical or pre-clinical studies with ABT-165 or bevacizumab and represents a significant improvement to current standard treatments with folinic acid and 5-fluorouracil.

We claim:

1. A method for the treatment of colorectal cancer in a subject who is in need thereof, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with folinic acid and 5-fluorouracil.

2. The method of claim 1, wherein ABT-165 is dosed once every 2 weeks in a subject.

3. The method of claim 1, wherein ABT-165 is dosed in combination with folinic acid, 5-fluorouracil, and irinotecan to a subject.

4. The method of claim 1, wherein ABT-165 is dosed at about 2.5 mg/kg every 2 weeks in combination with folinic acid, 5-fluorouracil, and irinotecan to a subject.

5. The method of claim 1, wherein ABT-165 is dosed at about 2.5 mg/kg every 2 weeks in combination with folinic acid, 5-fluorouracil, and irinotecan to a subject as first line therapy.

6. The method of claim 1, wherein ABT-165 is dosed at about 2.5 mg/kg every 2 weeks in combination with folinic acid, 5-fluorouracil, and irinotecan to a subject as second line or higher therapy.

7. A method for the treatment of cancer that is sensitive to FOLFIRI therapy, comprising administering to the subject an effective amount of a dual-variable domain immunoglobulin molecule with dual specificity for both delta-like ligand 4 (DLL4) and vascular endothelial growth factor (VEGF), ABT-165, dosed at about 2.5 mg/kg in combination with folinic acid and 5-fluorouracil, wherein said cancer is selected from the group consisting of: gastroesophageal cancer, pancreatic cancer, breast cancer, glioblastoma multiforme, ovarian cancer, or non-small cell lung cancer in a subject who is in need thereof.

8. The method of claim 7, wherein ABT-165 is dosed once every 2 weeks in a subject.

9. The method of claim 7, wherein ABT-165 is dosed in combination with folinic acid, 5-fluorouracil, and irinotecan to a subject.

10. The method of claim 7, wherein ABT-165 is dosed at about 2.5 mg/kg every 2 weeks in combination with folinic acid, 5-fluorouracil, and irinotecan to a subject.

11. The method of claim 7, wherein ABT-165 is dosed at about 2.5 mg/kg every 2 weeks in combination with folinic acid, 5-fluorouracil, and irinotecan to a subject as first line therapy.

12. The method of claim 7, wherein ABT-165 is dosed at about 2.5 mg/kg every 2 weeks in combination with folinic acid, 5-fluorouracil, and irinotecan to a subject as second line or higher therapy.


SEQUENCE LISTING (16 sequences)

SEQ ID NO: 1 - protein, 11 aa, Artificial
  Synthetic Peptide
RASEDIYSNLA

SEQ ID NO: 2 - protein, 7 aa, Artificial
  Synthetic Peptide
DTNNLAD

SEQ ID NO: 3 - protein, 9 aa, Artificial
  Synthetic Peptide
QQYNNYPPT

SEQ ID NO: 4 - protein, 11 aa, Artificial
  Synthetic Peptide
SASQDISNYLN

SEQ ID NO: 5 - protein, 7 aa, Artificial
  Synthetic Peptide
FTSSLHS

SEQ ID NO: 6 - protein, 8 aa, Artificial
  Synthetic Peptide
QQYSTVPW

SEQ ID NO: 7 - protein, 10 aa, Artificial
  Synthetic Peptide
GFTFSNFPMA

SEQ ID NO: 8 - protein, 16 aa, Artificial
  Synthetic Peptide
ISSSDGTTYYRDSVKG

SEQ ID NO: 9 - protein, 9 aa, Artificial
  Synthetic Peptide
GYYNSPFAY

SEQ ID NO: 10 - protein, 11 aa, Artificial
  Synthetic Peptide
SGYTFTNYGMN

SEQ ID NO: 11 - protein, 16 aa, Artificial
  Synthetic Peptide
INTYTGEPTYAADFKR

SEQ ID NO: 12 - protein, 14 aa, Artificial
  Synthetic Peptide
YPHYYGSSHWYFDV

SEQ ID NO: 13 - protein, 228 aa, Artificial
  Synthetic Peptide
DIQMTQSPSSLSASVGDRVTITCRASEDIYSNLAWYQQKPGKAPKLLIYDTNNLADGVPSRFSGSGSGTDFTLTISSLQPEDFATYYCQQYNNYPPTFGQGTKLEIKRTVAAPSVFIFPPDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 14 - protein, 247 aa, Artificial
  Synthetic Peptide
EVQLVESGGGLVQPGGSLRLSCAASGFTFSNFPMAWVRQAPGKGLEWVATISSSDGTTYYRDSVKGRFTISRDNAKNSLYLQMNSLRAEDTAVYYCARGYYNSPFAYWGQGTLVTVSSASTKGPEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 15 - protein, 334 aa, Artificial
  Synthetic Peptide
DIQMTQSPSSLSASVGDRVTITCRASEDIYSNLAWYQQKPGKAPKLLIYDTNNLADGVPSRFSGSGSGTDFTLTISSLQPEDFATYYCQQYNNYPPTFGQGTKLEIKRTVAAPSVFIFPPDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 577 aa, Artificial
  Synthetic Peptide
EVQLVESGGGLVQPGGSLRLSCAASGFTFSNFPMAWVRQAPGKGLEWVATISSSDGTTYYRDSVKGRFTISRDNAKNSLYLQMNSLRAEDTAVYYCARGYYNSPFAYWGQGTLVTVSSASTKGPEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK